(19) United States
(12) Patent Application Publication
(10) Pub. No.: US 2025/0246094 A1
(43) Pub. Date: Jul. 31, 2025

(54) INFORMATION PROCESSING APPARATUS, INFORMATION PROCESSING METHOD, AND PROGRAM

(71) Applicant: SONY GROUP CORPORATION, TOKYO (JP)

(72) Inventor: NAOYA SAZUKA, TOKYO (JP)

(21) Appl. No.: 18/855,813

(22) PCT Filed: Apr. 4, 2023
(86) PCT No.: PCT/JP2023/013885

§ 371 Date: Oct. 10, 2024

(30) Foreign Application Priority Data
Apr. 19, 2022 (JP) 2022-068609

(51) Int. Cl. G09B 19/00; A61B 5/00; A61B 5/16

(52) U.S. Cl. CPC G09B 19/00; A61B 5/165; A61B 5/742

(57) ABSTRACT

The present technology relates to an information processing apparatus, an information processing method, and a program capable of presenting a detection condition of a mindful state as visualized information.

An information processing apparatus includes an obtaining unit that obtains biological information regarding a user, a detection unit that analyzes the biological information obtained by the obtaining unit and that detects a mindful state of the user, and a generation unit that generates image data regarding a screen for presenting information regarding the mindful state detected by the detection unit to the user. The present technology can be applied to, for example, an information processing apparatus that presents, as visualized information, a detection condition of a mindful state to a student who is learning to enter the mindful state or a teacher.

### TECHNICAL FIELD

The present technology relates to an information processing apparatus, an information processing method, and a program, and for example, relates to an information processing apparatus, an information processing method, and a program capable of objectively evaluating a state during mindfulness.

### BACKGROUND ART

During these years, mindfulness, which realizes self-recognition, self-development, and the like through a psychological process for paying attention to internal or external experience of a person, is gaining attention. In response to such a background, devices that assist mindfulness have been proposed (see, for example, Patent Documents 1 and 2).

### CITATION LIST

### Patent Document

Patent Document 1: Japanese Patent Application Laid-Open No. 2020-103879
Patent Document 2: Japanese Patent Application Laid-Open No. 2019-208876

### SUMMARY OF THE INVENTION

### Problems to be Solved by the Invention

It is desired to appropriately present a result of detection of a mindful state to a student who is learning mindfulness or a teacher.

The present technology has been made in view of such circumstances, and an object thereof is to appropriately present a result of detection of a mindful state.

### Solutions to Problems

An information processing apparatus according to an aspect of the present technology is an information processing apparatus including an obtaining unit that obtains biological information regarding a user, a detection unit that analyzes the biological information obtained by the obtaining unit and that detects a mindful state of the user, and a generation unit that generates image data regarding a screen for presenting information regarding the mindful state detected by the detection unit to the user.

An information processing method according to another aspect of the present technology is an information processing method used by an information processing apparatus. The information processing method includes obtaining biological information regarding a user, analyzing the obtained biological information and detecting a mindful state of the user, and generating image data regarding a screen for presenting information regarding the detected mindful state to the user.

A program according to another aspect of the present technology is a program for causing a computer to execute a process including the steps of obtaining biological information regarding a user, analyzing the obtained biological information and detecting a mindful state of the user, and generating image data regarding a screen for presenting information regarding the detected mindful state to the user.

In the information processing apparatus, the information processing method, and the program according to the aspects of the present technology, biological information regarding a user is obtained, the obtained biological information is analyzed, a mindful state of the user is detected, and image data regarding a screen that presents information regarding the detected mindful state to the user is generated.

Note that the information processing apparatus may be an independent apparatus or may be an internal block including constituting an apparatus.

Note that a program to be provided may be transmitted via a transmission medium or be stored in a storage medium.

### BRIEF DESCRIPTION OF DRAWINGS

FIG. 1 is a diagram illustrating a configuration of an information processing system according to an embodiment, to which the present technology is applied.

FIG. 2 is a diagram illustrating a configuration example of the information processing apparatus.

FIG. 3 is a diagram illustrating functions of the information processing apparatus.

FIG. 4 is a flowchart for describing operation of the information processing apparatus.

FIG. 5 is a diagram for describing a first screen.

FIG. 6 is a diagram illustrating another graph displayed in a first graph display part.

FIG. 7 is a diagram for describing a second screen.

FIG. 8 is a diagram for describing a third screen.

FIG. 9 is a diagram for describing a fourth screen.

FIG. 10 is a diagram for describing a fifth screen.

FIG. 11 is a diagram for describing a sixth screen.

FIG. 12 is a diagram for describing a seventh screen.

FIG. 13 is a diagram for describing notification timing.

FIG. 14 is a diagram for describing an eighth screen.

FIG. 15 is a diagram for describing a ninth screen.

FIG. 16 is a diagram for describing a tenth screen.

FIG. 17 is a diagram for describing an eleventh screen.

FIG. 18 is a diagram for describing a twelfth screen.

FIG. 19 is a diagram for describing a thirteenth screen.

FIG. 20 is a diagram for describing a fourteenth screen.

FIG. 21 is a diagram for describing a fifteenth screen.

FIG. 22 is a diagram for describing a sixteenth screen.

FIG. 23 is a diagram for describing a seventeenth screen.

FIG. 24 is a diagram for describing an eighteenth screen.

FIG. 25 is a diagram for describing a nineteenth screen.

### MODE FOR CARRYING OUT THE INVENTION

A mode (hereinafter referred to as an embodiment) for implementing the present technology will be described hereinafter.

### Configuration of Information Processing System

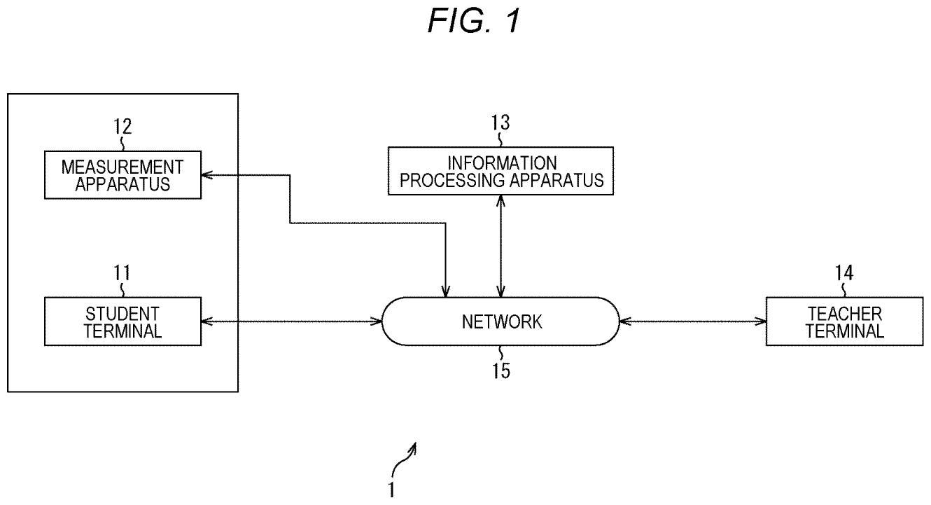

FIG. 1 is a diagram illustrating a configuration of an information processing system according to an embodiment, to which the present technology is applied.

An information processing system 1 illustrated in FIG. 1 includes a student terminal 11, a measurement apparatus 12, an information processing apparatus 13, a teacher terminal 14, and a network 15. The network 15 is a communication network represented by the Internet, and achieves mutual communication between the student terminal 11, the measurement apparatus 12, the information processing apparatus 13, and the teacher terminal 14.

The information processing system 1 illustrated in FIG. 1 is a system that detects that a student A is in a mindful state and feeds back a result of the detection to the student A. There are cases where the feedback is directly given to the student A and cases where a teacher gives an instruction to the student A after checking a result of the feedback. As described later, the information processing system 1 is a system suitable for use by a user such as the student A or the teacher in learning mindfulness and making progress.

The student terminal 11 is a terminal owned by the student A, and is a smartphone, a tablet terminal, a personal computer (PC), a smart watch, or the like.

The measurement apparatus 12 is a sensor or a predetermined apparatus that obtains biological information regarding the student A, which is used to detect a mindful state. The measurement apparatus 12 is, for example, a heart rate monitor, an electroencephalograph, a sphygmomanometer, a thermometer, a sweat sensor (an electrodermal monitor that measures an electrodermal potential) that measures an amount of perspiration, an electroencephalograph that measures brain waves, or the like. By using one or a plurality of these biological sensors that obtain information regarding a living body, a mindful state is detected. These sensors may be sensors to be worn on the head (head sensors), spectacle type sensors (eye glass sensors), sensors to be worn on the wrist (wrist sensors), or the like, and may be worn on any body part.

The measurement apparatus 12 may include an imaging device. By analyzing an image obtained from the imaging device, for example, a complexion, a facial expression, movement, a voice (breathing sound), and the like of the student A may be analyzed, and an analysis result may be used to detect a mindful state.

As the measurement apparatus 12, functional magnetic resonance imaging (FMRI) may be used, instead. The following document, for example, proposes detection of a mindful state based on FMRI.

Weng, H. Y., Feldman, J. L., Leggio, L., Napadow, V., Park, J., & Price, C. J. (2021). Interventions and manipulations of interoception. Trends in Neurosciences, 44 (1), 52-62.

The measurement apparatus 12 may be an apparatus integrated with the student terminal 11. For example, the measurement apparatus 12 may be an apparatus such as a smart watch, and a sensor provided in a smart watch may be used as the measurement apparatus 12, instead.

The information processing apparatus 13 obtains a result of measurement from the measurement apparatus 12 over the network 15, determines whether or not the student A is in a mindful state, and generates image data based on a result of the determination. Although details will be described later, for example, statistics are taken while a mindful state is established, and image data regarding an image of a graph or a table indicating statistical results is generated.

The image data generated by the information processing apparatus 13 is supplied to the student terminal 11 and displayed on a display (display unit) of the student terminal 11. The student A can recognize his/her own mindful state by viewing the graph or the table displayed on the display unit, and can use the graph or the table as a material for achieving a better mindful state.

The image data generated by the information processing apparatus 13 is also supplied to the teacher terminal 14. The teacher terminal 14 is a terminal owned by the teacher who instructs the student A, and the teacher can view the graph or the table indicating the mindful state of the student A and use the graph or the table as a reference for teaching.

There may be one student and one teacher, or may be a plurality of students and one teacher. FIG. 1 illustrates the student A and the teacher (the student terminal 11 and the teacher terminal 14), but a plurality of student terminals 11 and a plurality of teacher terminals 14 are connected to the network 15.

The functions of the information processing apparatus 13 may be included in the student terminal 11 and the teacher terminal 14, and the student terminal 11 and the teacher terminal 14 may be configured to perform a process including obtaining and analyzing a result of measurement from the measurement apparatus 12 and generating and displaying image data. As described above, the measurement apparatus 12 may be included in the student terminal 11, and in the case of such a configuration, may be configured to perform a process including performing measurement using the measurement apparatus 12, analyzing a result of the measurement, and generating and displaying image data.

In the following description, a case will be mainly described as an example where the information processing system 1 has the configuration as illustrated in FIG. 1 and the information processing apparatus 13 obtains and analyzes a result of measurement from the measurement apparatus 12 and generates image data.

### Configuration of Information Processing Apparatus

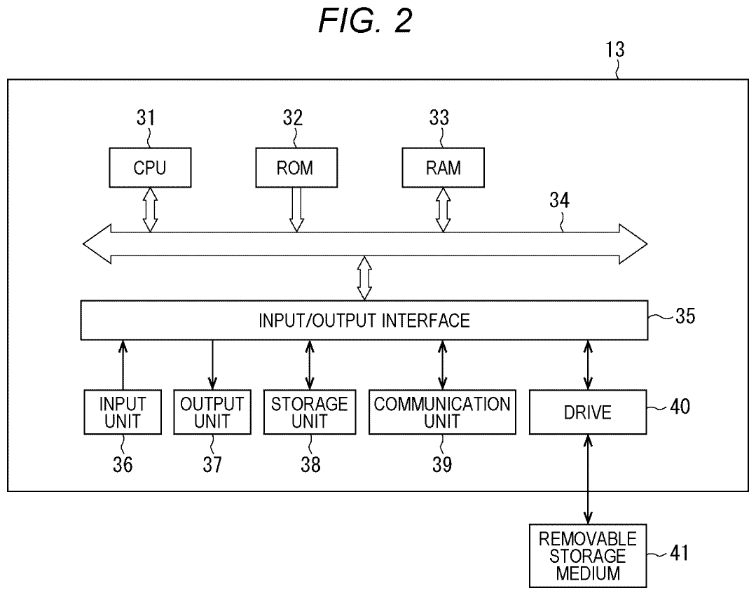

FIG. 2 is a diagram illustrating a configuration example of the information processing apparatus 13. The information processing apparatus 13 may be achieved by a personal computer.

In the information processing apparatus 13, a central processing unit (CPU) 31, a read-only memory (ROM) 32, and a random-access memory (RAM) 33 are connected to one another by a bus 34. An input/output interface 35 is also connected to the bus 34. An input unit 36, an output unit 37, a storage unit 38, a communication unit 39, and a drive 40 are connected to the input/output interface 35.

The input unit 36 includes a keyboard, a mouse, a microphone, and the like. The output unit 37 includes a display, a speaker, and the like. The storage unit 38 includes a hard disk, a nonvolatile memory, and the like. The communication unit 39 includes a network interface and the like. The drive 40 drives a removable storage medium 41 such as a magnetic disk, an optical disc, a magneto-optical disk, or a semiconductor memory.

In the information processing apparatus 13 configured as described above, for example, the CPU 31 loads a program stored in the storage unit 38 into the RAM 33 via the input/output interface 35 and the bus 34 and executes the program to perform the above-described process.

The program executed by the information processing apparatus 13 (CPU 31) may be stored in the removable storage medium 41 as a package medium or the like, for example, and provided. Furthermore, the program may be provided via a wired or wireless transmission medium such as a local area network, the Internet, or digital satellite broadcasting.

In the information processing apparatus 13, the program may be installed in the storage unit 38 via the input/output interface 35 by attaching the removable storage medium 41 to the drive 40. Furthermore, the program may be received by the communication unit 39 via a wired or wireless transmission medium and installed in the storage unit 38. In addition, the program may be installed in the ROM 32 or the storage unit 38 in advance.

The information processing apparatus 13 may obtain a result of measurement by the measurement apparatus 12 via the communication unit 39, or may obtain a result of measurement by the measurement apparatus 12 stored on the removable storage medium 41.

The student terminal 11 and the teacher terminal 14 can also have a configuration similar to that of the information processing apparatus 13 illustrated in FIG. 2.

### Functions of Information Processing Apparatus

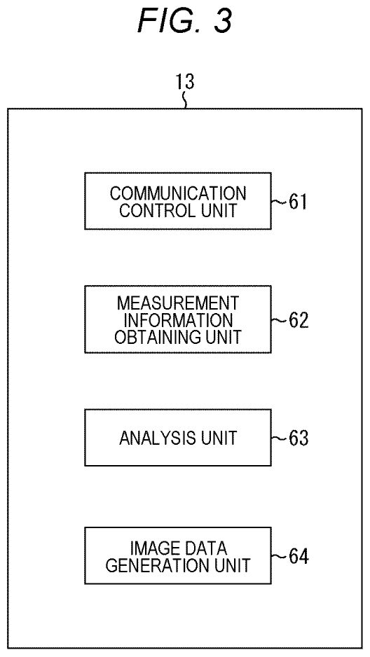

FIG. 3 is a diagram illustrating the functions of the information processing apparatus 13. The information processing apparatus 13 includes a communication control unit 61, a measurement information obtaining unit 62, an analysis unit 63, and an image data generation unit 64.

The communication control unit 61 controls the communication unit 39 (FIG. 2) to control communication with other apparatuses over the network 15. The measurement information obtaining unit 62 obtains measurement information obtained from the measurement apparatus 12 over the network 15 under the control of the communication control unit 61.

The analysis unit 63 analyzes the measurement information obtained by the measurement information obtaining unit 62 and detects a mindful state of the student A. Here, a case will be described as an example in which two states, namely a mindful state and states other than a mindful state (hereinafter referred to as other states), are detected.

The present technology can be applied to not only a case where two states are detected but also a case where a plurality of states is detected. For example, three states, namely a deeply mindful state, a moderately mindful state, and a shallowly mindful state, may be provided, and which one of these states a current state is, may be detected. Other states may also be divided into a plurality of states, namely a state in which the student A is interested in another thing, a state in which there is an external stimulus, and the like, for example, and one of these states may be detected.

In a case where the measurement apparatus 12 employs FMRI, for example, the analysis unit 63 analyzes information obtained through the FMRI and detects a mindful state of the student A. In a case where the measurement apparatus 12 is a sensor that measures brain waves or respiration, the analysis unit 63 detects a mindful state of the student A using information obtained from the sensor. In this case, the analysis unit 63 may include a learning model obtained through machine learning and be configured to detect a mindful state of the student A using the learning model and the information from the sensor.

For example, the analysis unit 63 may detect, using the measurement information obtained from the measurement apparatus 12, whether or not the student is concentrating on respiration, and if it can be detected that the student is concentrating on respiration, output an analysis result indicating that a mindful state has been detected.

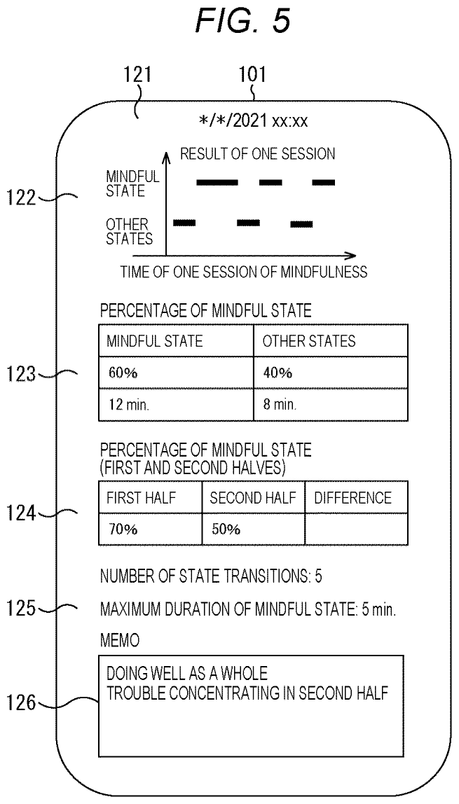

The image data generation unit 64 generates image data regarding a screen to be presented to the student A and the teacher, which will be described with reference to FIG. 5 and subsequent drawings. The generated image data is supplied to the student terminal 11 and/or the teacher terminal 14 under the control of the communication control unit 61.

Each function illustrated in FIG. 3 may be included in the student terminal 11, instead. In a case where the student terminal 11 has each function illustrated in FIG. 3, the student terminal 11 itself obtains and analyzes the measurement information from the measurement apparatus 12, generates image data, and presents a screen based on the image data to the student A.

In a case where the teacher terminal 14 has each function illustrated in FIG. 3, the teacher terminal 14 obtains and analyzes the measurement information from the measurement apparatus 12, generates image data, and presents a screen based on the image data to the teacher. The image data may be generated such that the screen becomes suitable for the teacher.

### Operation of Information Processing Apparatus

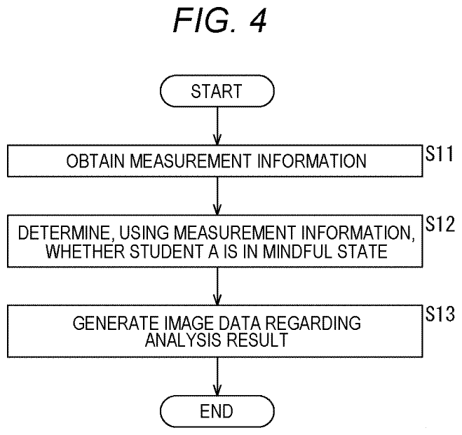

Operation of the information processing apparatus 13 having the functions illustrated in FIG. 3 will be described with reference to a flowchart of FIG. 4.

In step S11, the information processing apparatus 13 obtains measurement information from the measurement apparatus 12. Under the control of the communication control unit 61, the measurement information obtaining unit 62 obtains measurement information necessary to detect a mindful state of the student A supplied from the measurement apparatus 12 over the network 15.

In step S12, the analysis unit 63 of the information processing apparatus 13 uses the measurement information obtained by the measurement information obtaining unit 62 to determine whether or not the student A is in a mindful state.

In step S13, the image data generation unit 64 generates, using an analysis result obtained by the analysis unit 63 (a determination result indicating whether or not a mindful state has been established), image data regarding a screen including a graph and a table to be provided for the student A and the teacher.

A screen based on image data generated through such processing will be described with reference to FIG. 5 and subsequent drawings. The screen based on the image data is a screen in which information regarding a mindful state is displayed as described later. The information regarding a mindful state includes information such as a period of time for which the mindful state has been established and percentage of the period of time and also includes a proficiency level for mindfulness.

### Examples of Screens

FIG. 5 is a diagram illustrating an example of a screen (referred to as a first screen as appropriate) displayed on the display unit 101 of the student terminal 11. Here, the description will be continued assuming that the screen is displayed on the student terminal 11, but the screen is also displayed on the teacher terminal 14. That is, the same screen is displayed on the student terminal 11 and the teacher terminal 14. As described later, a screen suitable for the teacher, that is, a screen different from that displayed on the student terminal 11 may be displayed on the teacher terminal 14.

The display unit 101 includes a display of the student terminal 11, and also functions as a display unit that displays a user interface (UI).

Referring to the example of the screen illustrated in FIG. 5, a date display part 121, a first graph display part 122, a first table display part 123, a second table display part 124, a first information display part 125, and a first memo input part 126 are provided in this order from a top of the display unit 101.

The date display part 121 displays a date and a time when a mindful state was measured. For example, in a case where the student A performs learning for maintaining a mindful state, a date of the learning is displayed.

In the first graph display part 122, a horizontal axis represents time and a vertical axis represents the two states. In the two states, an upper side of a graph represents a mindful state, and a lower side of the graph represents the other states. The graph displayed in the first graph display part 122 is a graph in which horizontal bars indicate periods of time for which a mindful state continued and periods of time for which the other states continued.

It can be seen from the graph illustrated in FIG. 5 that a predetermined time had elapsed while switching between the mindful state and the other states, that is, one of the other states was established first, then the mindful state continued for a certain period of time, and then one of the other states was established and continued for a certain period of time.

Although time points are not described on the horizontal axis of the first graph display part 122 in the example illustrated in FIG. 5, time points may be described, instead. The time points to be displayed may be time points when the learning was actually performed, or may be elapsed time with a start time of the learning set to 0. In the example illustrated in FIG. 5, the periods of time for which the mindful state continued and the periods of time for which the other states continued are summarized in tables displayed below the first graph display part 122, and the tables can be referred to.

In the first graph display part 122 illustrated in FIG. 5, a graph displayed in a case where the two states, namely, the mindful state and the other states, are detected is shown. For example, a plurality of mindful states such as a deeply mindful state and a shallowly mindful state may be detected, and a graph indicating a state of transition between the plurality of states may be displayed in the first graph display part 122, instead.

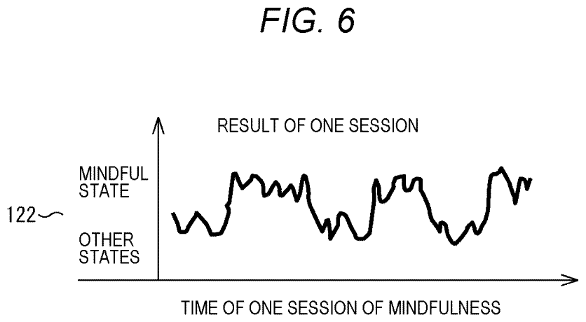

FIG. 6 is a diagram illustrating an example of another graph displayed in the first graph display part 122. The graph illustrated in FIG. 6 is an example of a graph in a case where the mindful state and the other states can be finely detected and represented as continuous values. According to the graph illustrated in FIG. 6, for example, fine transitions in the mindful state can be grasped. The student A refers to the graph illustrated in FIG. 6 and can perform learning such that, for example, parts of the graph in the mindful state become flatter, that is, the mindful state is maintained.

The first table display part 123 displays a table indicating a total time for which the mindful state continued, percentage of the total time, a total time for which the other states continued, and percentage of the total time. In the table in the first table display part 123 illustrated in FIG. 5, it is described that there was 12 minutes of the mindful state and the percentage of the mindful state was 60% and that there was 8 minutes of the other states and the percentage of the other states was 40%.

It can be seen that, in the learning at this time, learning time is 20 minutes, and the student A is in the mindful state for 60% of the learning time, namely 12 minutes, and not in the mindful state for 40% of the learning time, namely 8 minutes. The learning time corresponds to a period of time for which the measurement apparatus 12 measured biological information regarding the student A in order to obtain the biological information.

The information regarding the percentage of the mindful state in a predetermined period (corresponds to the learning time in this case) for which whether or not the mindful state was established was measured is thus displayed. Note that although a case where learning for maintaining a mindful state is performed has been described here as an example, the present technology can also be applied to a case where a mindful state in a predetermined period other than a time of learning is measured.

The second table display part 124 displays a table in which the total time for which the mindful state continued is divided into a first half and a second half, and the percentage of the mindful state in each of the first and second halves is described. Since the total time described in the table in the first table display part 123 is divided into the first half and the second half in the table in the second table display part 124 illustrated in FIG. 5, it can be seen that the student A was in the mindful state for 70% of the first 10 minutes of 20 minutes and 50% of the last 10 minutes of 20 minutes.

Text-based information is described in the first information display part 125. In the example illustrated in FIG. 5, information indicating that the number of state transitions was 5 and information indicating that maximum duration of the mindful state was 5 minutes are described. The number of state transitions is a sum of the number of transitions from the other states to the mindful state and the number of transitions from the mindful state to the other states, and is a value indicating that, when small, the mindful state could be maintained for a long time and progress was made.

The longer the duration of the mindful state, the better. By looking at the information displayed in the first information display part 125, the student A can consider how to make the number of state transitions smaller and the duration of the mindful state longer.

The first memo input part 126 is provided for the student A to input their subjective thought. The student A inputs, to the first memo input part 126, what they have subjectively found about their own state, such as whether or not the learning of mindfulness was done well. By comparing the input in the first memo input part 126 with what is displayed in each of the first graph display part 122, the first table display part 123, the second table display part 124, and the first information display part 125, the student A can compare their own recognition with an actual result and find points in common and differences, which are useful for next learning.

The student A may input, to the first memo input part 126, what they think with reference to the graph or the table displayed above the first memo input part 126 by looking at the information, or in a case where the student A feels differently from the information, they may write the feeling. The first memo input part 126 may be displayed in a state where no graph or table is displayed, and order of display and a manner of display may be appropriately changed so that the student A can first write what they feel after the learning without referring to the information obtained from the graph or the table.

Aside from the first memo input part 126, the example of the first screen illustrated in FIG. 5 is an example, and is not description indicating limitation. Examples of screens described below, too, are not description indicating limitation, and arrangement, size, information to be displayed, the order of display, and the like may be changed as appropriate.

### Second Screen

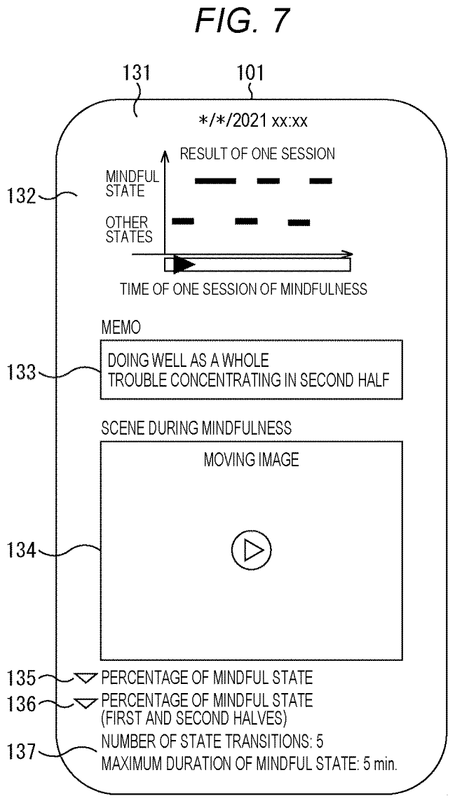

FIG. 7 is a diagram illustrating an example of a screen (referred to as a second screen as appropriate) displayed on the display unit 101 of the student terminal 11. The second screen illustrated in FIG. 7 may be a screen that can be browsed by scrolling the first screen illustrated in FIG. 5, or may be a screen presented as a screen different from the first screen. Examples of screens described below, too, may also be screens presented alone or screens presented continuously through scrolling or the like.

The second screen illustrated in FIG. 7 includes, in order from the top, a date display part 131, a second graph display part 132, a second memo input part 133, a first moving image display part 134, a third table display part 135, a fourth table display part 136, and a second information display part 137.

The date display part 131 displays a date and a time when a mindful state was detected (learned).

The graph described as being displayed in the first graph display part 122 of the first screen (FIG. 5) is displayed in the second graph display part 132. Since the first moving image display part 134 that reproduces a moving image captured during the learning is provided on the second screen, a seek bar is displayed below the graph displayed in the second graph display part 132 (below the horizontal axis). By moving a cursor (a triangular shape in the drawing) displayed on the seek bar, the student A can view a desired part of the moving image, that is, for example, a part of the moving image at a time when the student A was in the mindful state.

The first moving image display part 134 displays the moving image obtained by shooting the student A during the learning. For example, a playback mark is displayed in the first moving image display part 134, and the moving image is played back when the playback mark is used. As described above, a part that is being played back is indicated by a position of the cursor on the seek bar displayed in the second graph display part 132, and the student A can recognize whether a part of the moving image in the mindful state or a part of the moving image in the other states is being played back by checking the position of the cursor.

A second memo input part 133 is provided between the second graph display part 132 and the first moving image display part 134. As with the first memo input part 126 in the first screen (FIG. 5), the second memo input part 133 is provided as a field to which the student A inputs their subjective thoughts. As described above, the second memo input part 133 may be displayed at any position in the screen.

A third table display part 135, a fourth table display part 136, and a second information display part 137 are provided below the first moving image display part 134. The third table display part 135 and the fourth table display part 136 correspond to the first table display part 123 and the second table display part 124 on the first screen (FIG. 5), respectively, but are folded on the second screen illustrated in FIG. 7 and the tables are not displayed.

In a case where the student A desires to display a table, the student A uses an expansion button (a button indicated by a triangular shape in the drawing) displayed to the left of text “percentage of mindful state” in the drawing. By using the expansion button, for example, a table similar to that displayed in the first table display part 123 in FIG. 5 is displayed. In a case where the student A desires to display a graph, the student A uses a corresponding expansion button (a button indicated by a triangular shape in the drawing) displayed to the left of text “percentage of mindful state (first and second halves)” in the drawing. By using the expansion button, for example, a table similar to that displayed in the second table display part 124 in FIG. 5 is displayed.

It is also possible to employ a configuration in which the text “percentage of mindful state” and the text “percentage of mindful state (first and second halves)” are used as expansion buttons, and the tables may be displayed in a case where these texts are used.

The second information display part 137 corresponds to the first information display part 125 in the first screen (FIG. 5), and displays the number of state transitions, which indicates the number of transitions between the mindful state and the other states, and the maximum duration of the mindful state.

As in the second screen, a moving image may be displayed, or graphs and tables may be expanded in accordance with instructions from the student A.

### Third Screen

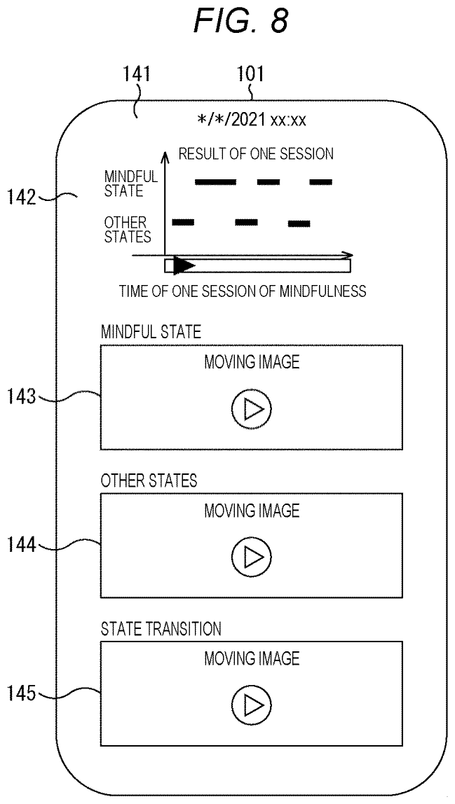

FIG. 8 is a diagram illustrating an example of a screen (referred to as a third screen as appropriate) displayed on the display unit 101 of the student terminal 11.

The third screen illustrated in FIG. 8 includes, in order from the top, a date display part 141, a third graph display part 142, a second moving image display part 143, a third moving image display part 144, and a fourth moving image display part 145.

The date display part 141 displays a date and a time when a mindful state was detected (learned).

The graph described as being displayed in the second graph display part 132 of the second screen (FIG. 7) is displayed in the third graph display part 142.

In the third screen illustrated in FIG. 8, three moving image display parts are provided. The second moving image display part 143 that displays a moving image in the mindful state, the third moving image display part 144 that displays a moving image in the other states, and the fourth moving image display part 145 that displays a moving image in a transition state are provided.

Like the third screen illustrated in FIG. 8, a screen on which moving images are mainly displayed may be presented to the student A or the teacher. By providing areas for playing back moving images in different states, for example, the different states can be viewed, compared, and examined.

### Fourth Screen

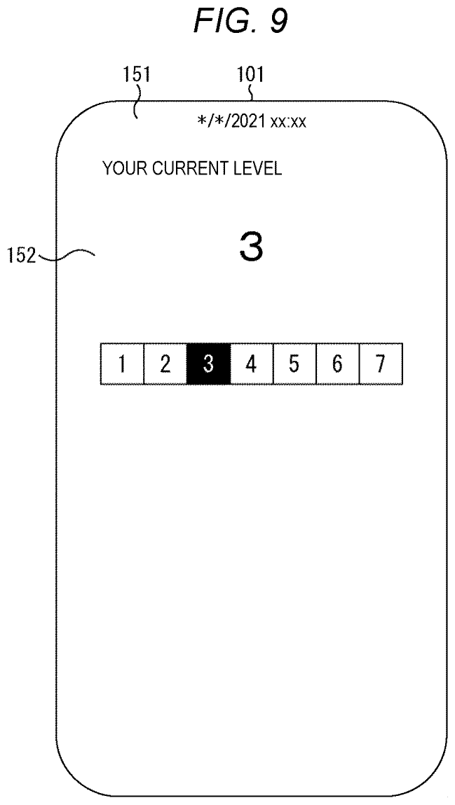

FIG. 9 is a diagram illustrating an example of a screen (referred to as a fourth screen as appropriate) displayed on the display unit 101 of the student terminal 11.

The fourth screen illustrated in FIG. 9 includes a date display part 151 and a level display part 152. The date display part 151 displays a date and a time when a mindful state was detected (learned).

The level display part 152 displays a level calculated from various parameters provided for the student A as a table, a graph, information, or the like. Examples of the parameters include periods of time and percentage of the mindful state and the other states in learning time, the number of transitions, and the maximum duration of mindfulness. A level obtained by comprehensively judging these parameters is displayed in the level display part 152.

For example, in a case where the percentage of the mindful state is converted into a level and the level is 3, “3” is displayed in the level display part 152 as illustrated in FIG. 9. A gauge with numbers 1 to 7 is displayed below the value in the level display part 152 so that the score of the level can be distinguished from other scores. FIG. 9 indicates that the score is 3 on a 7-point scale. This evaluation may be used as information indicating the proficiency level of the student A. In this case, it is indicated that the proficiency level has seven stages and the student A has reached a third stage.

By viewing the fourth screen, the student A can grasp their own level and consider how to increase the level from the information displayed on the first to third screens. The fourth screen may also be presented to the teacher, and the teacher can know the level of the student A by viewing the fourth screen, and can use the fourth screen as a reference for how to teach the student A.

### Fifth Screen

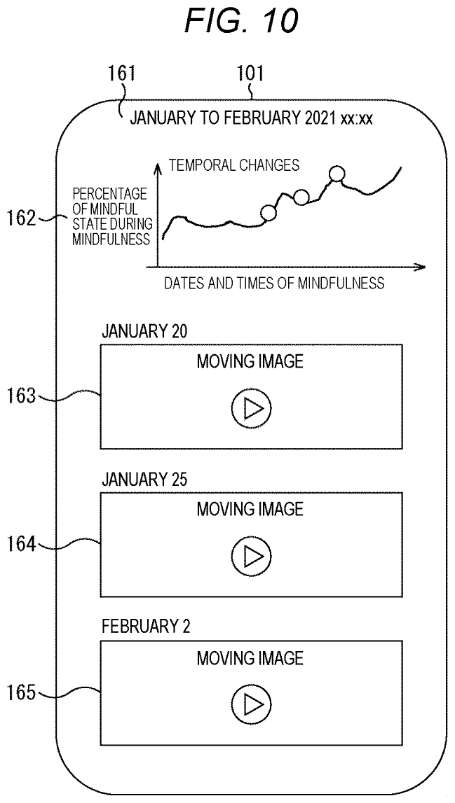

FIG. 10 is a diagram illustrating an example of a screen (referred to as a fifth screen as appropriate) displayed on the display unit 101 of the student terminal 11.

The fifth screen illustrated in FIG. 10 is an example of a screen in a case where, after a plurality of times of learning related to mindfulness is performed, information regarding the learning is collectively displayed. The fifth screen illustrated in FIG. 10 includes, in order from the top, a date display part 161, a fourth graph display part 162, a fifth moving image display part 163, a sixth moving image display part 164, and a seventh moving image display part 165.

In the date display part 161, a period for which information is displayed as the fifth screen among periods during which learning was performed is displayed. For example, as in the example illustrated in FIG. 10, a period such as January to February is displayed.

The fourth graph display part 162 displays a graph in which a horizontal axis represents dates and times when mindfulness was performed and a vertical axis represents the percentage of the mindful state during mindfulness. For example, a graph displayed in the fourth graph display part 162 is generated using the information regarding the table displayed in the first table display part 123 of the first screen (FIG. 5).

From the graph displayed in the fourth graph display part 162, the student A and the teacher can recognize how the percentage of the mindful state changed through repeated learning. For example, a degree of progress can be checked on the basis of how steeply the percentage of the mindful state increased. Circular marks on the graph are displayed at positions corresponding to dates and times of moving images displayed in the fifth moving image display part 163, the sixth moving image display part 164, and the seventh moving image display part 165.

In the example illustrated in FIG. 10, a moving image of learning on January 20 is displayed in the fifth moving image display part 163, a moving image of learning on January 25 is displayed in the sixth moving image display part 164, and a moving image of learning on February 2 is displayed in the seventh moving image display part 165. The displayed moving image may be any of moving images in the mindful state, moving images in the other states, and moving images in the transition state.

By presenting the moving images to the student A and the teacher, it is possible to compare details of a plurality of situations measured in the past. For example, it is possible to compare mindful states at different dates and times while viewing the moving images, and it is possible to consider improvement points for future learning.

### Sixth Screen

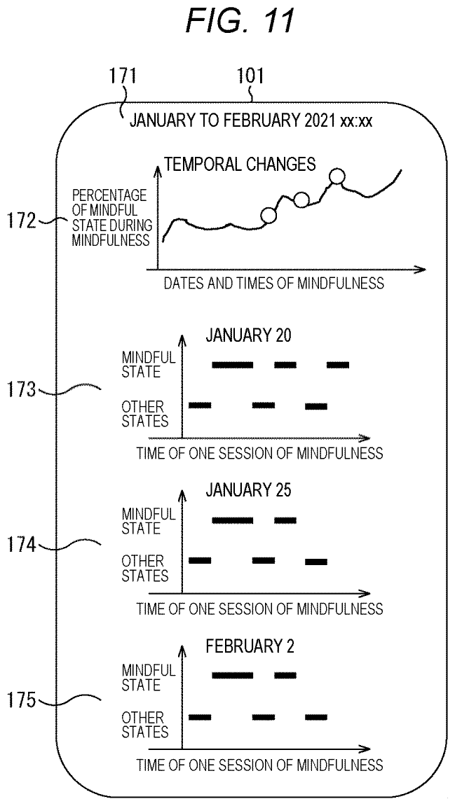

FIG. 11 is a diagram illustrating an example of a screen (referred to as a sixth screen as appropriate) displayed on the display unit 101 of the student terminal 11.

The sixth screen illustrated in FIG. 11 is, as with the fifth screen (FIG. 10), an example of a screen in a case where, after a plurality of times of learning related to mindfulness is performed, information regarding the learning is collectively displayed. The sixth screen illustrated in FIG. 11 includes, in order from the top, a date display part 171, a fifth graph display part 172, a sixth graph display part 173, a seventh graph display part 174, and an eighth graph display part 175.

In the date display part 171, a period for which information is displayed as the sixth screen among periods during which learning was performed is displayed. For example, as in the example illustrated in FIG. 10, a period such as January to February is displayed.

The graph described as being displayed in the fourth graph display part 162 of the fifth screen (FIG. 10) is displayed in the fifth graph display part 172. By viewing the graph displayed in the fifth graph display part 172, the student A and the teacher can check the degree of progress on the basis of how steeply the percentage of the mindful state increased.

A graph of learning on January 20 is displayed in the sixth graph display part 173, a graph of learning on January 25 is displayed in the seventh graph display part 174, and a graph of learning on February 2 is displayed on the eighth graph display part 175. These graphs are the graph displayed in the first graph display part 122 of the first screen (FIG. 5).

By presenting the sixth screen to the student A and the teacher, it is possible to compare details of a plurality of situations measured in the past. For example, it is possible to compare mindful states at different dates and times, and it is possible to consider improvement points for future learning.

### Seventh Screen

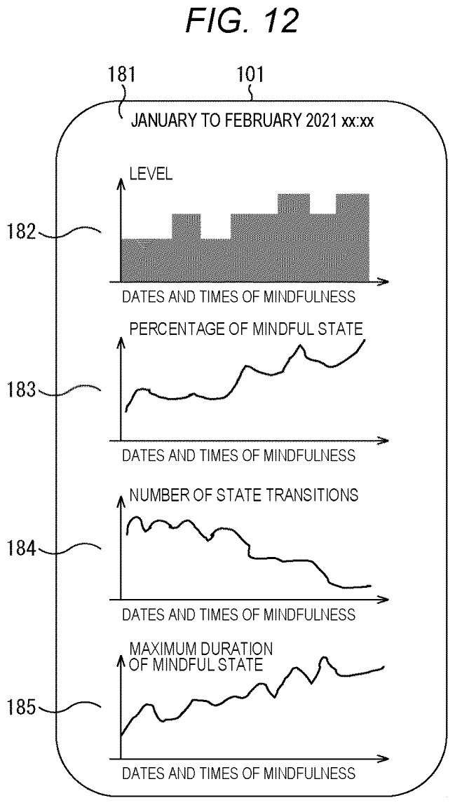

FIG. 12 is a diagram illustrating an example of a screen (referred to as a seventh screen as appropriate) displayed on the display unit 101 of the student terminal 11.

The seventh screen illustrated in FIG. 12 is, as with the fifth screen (FIG. 10), an example of a screen in a case where, after a plurality of times of learning related to mindfulness is performed, information regarding the learning is collectively displayed. The seventh screen illustrated in FIG. 12 includes, in order from the top, a date display part 181, a ninth graph display part 182, a tenth graph display part 183, an eleventh graph display part 184, and a twelfth graph display part 185.

In the date display part 181, a period for which information is displayed as the seventh screen among periods during which learning was performed is displayed. For example, as in the example illustrated in FIG. 12, a period such as January to February is displayed.

The ninth graph display part 182 displays a graph in which a horizontal axis represents dates and times when learning of mindfulness was performed and a vertical axis represents the level. The graph related to the level displayed in the ninth graph display part 182 is a graph in which the level presented to the student A and the teacher on the fourth screen (FIG. 9) is arranged on a day-to-day basis. From the graph related to the level displayed in the ninth graph display part 182, changes in the level can be seen at a glance, and the degree of progress can be checked.

The tenth graph display part 183 displays a graph in which a horizontal axis represents the dates and times when the learning of mindfulness was performed and a vertical axis represents the percentage of the mindful state. The graph displayed in the tenth graph display part 183 is a graph in which the degree of progress can be checked on the basis of how the percentage of the mindful state increased.

The eleventh graph display part 184 displays a graph in which a horizontal axis represents the dates and times when the learning of mindfulness was performed and a vertical axis represents the number of state transitions. The graph displayed in the eleventh graph display part 184 is generated using, for example, the information regarding the number of state transitions displayed in the first information display part 125 of the first screen (FIG. 5). The graph displayed in the eleventh graph display part 184 is a graph in which the degree of progress can be checked on the basis of how the number of state transitions decreased.

The twelfth graph display part 185 displays a graph in which a horizontal axis represents the dates and times when the learning of mindfulness was performed and a vertical axis represents the maximum duration of the mindful state. The graph displayed in the twelfth graph display part 185 is generated using, for example, the information regarding the maximum duration of the mindful state displayed in the first information display part 125 of the first screen (FIG. 5). The graph displayed in the twelfth graph display part 185 is a graph in which the degree of progress can be checked on the basis of how the maximum duration of the mindful state increased.

The screens that collectively display graphs indicating temporal changes in various parameters necessary for progress can thus be presented to the student A and the teacher. By referring to the seventh screen, the degree of progress can be examined from various angles, whether the student A is progressing or stagnating can be grasped, and improvement points for future learning can be considered.

### Real-time Notification

The first to seventh screens described above are screens presented after completion of learning. If a state transition occurs while the student A is learning mindfulness, the student A may be notified of the state transition.

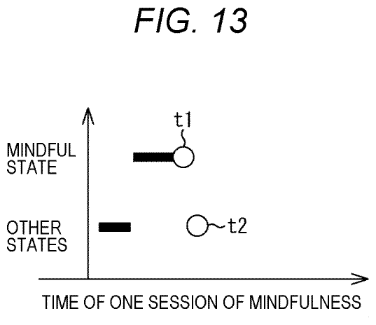

FIG. 13 is a diagram for describing a notification timing in a case where notification is performed for the student A in real-time. The graph illustrated in FIG. 13 is a part of the graph displayed in the first graph display part 122 of the first screen (FIG. 5). In a case where it is assumed that present time is a time t2, measurement has not yet been performed after the time t2.

At a time t1, a state transitions from the mindful state to another state. A time at which the transition to the other state has been completed is the time t2. The time t1 and the time t2 are the same time or slightly different times. The student A is notified of the transition of the state at a timing of the transition from the mindful state to the other state, that is, at the time t1 or the time t2 in this case.

In a case where a situation that is likely to transition from the mindful state to another state is detected and notification is performed to enable the student A to make an effort to remain in the mindful state in such a situation, the notification is performed at the time t1.

In a case where a situation in which the state has transitioned from the mindful state to another state is detected and notification is performed to enable the student A to make an effort to return to the mindful state in such a situation, the notification is performed at the time t2.

Since it can be assumed that eyes of the student A are closed while the student A is learning mindfulness, the notification is performed using sound or vibration. Notification in which sound and vibration are combined may be performed, or notification may be performed using only sound or vibration.

Such notification may be performed for the teacher, and the teacher may give an instruction to the student A after receiving the notification. Furthermore, in a case where the notification is performed for the teacher, a graph such as that illustrated in FIG. 13 may be presented to the teacher, and the teacher may give an instruction at an appropriate timing with reference to the presented graph.

As described above, a system that detects a state in real-time and that performs some notification can also be constructed.

### Screens for Teacher

The first to seventh screens have been described as screens presented to the student A and the teacher. A teacher might teach a student on a one-to-one basis or might teach a plurality of students. Screens to be presented to a teacher may be screens suitable for a teacher. Furthermore, screens for a student may also be screens suitable for a teacher in a case where there is a teacher.

### Eighth Screen

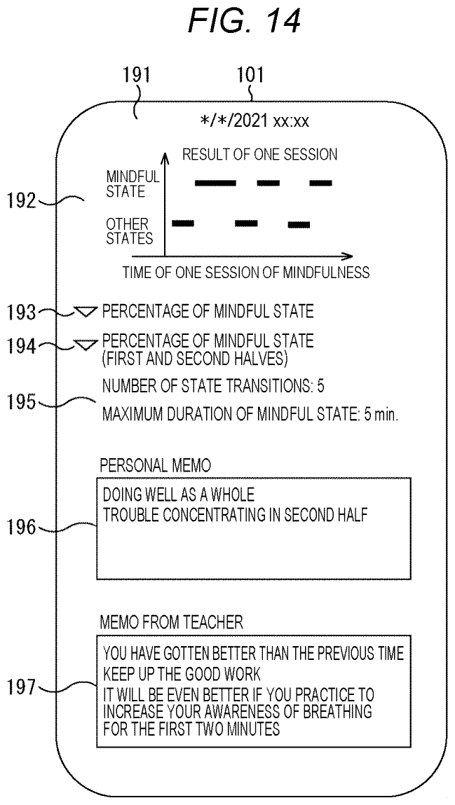

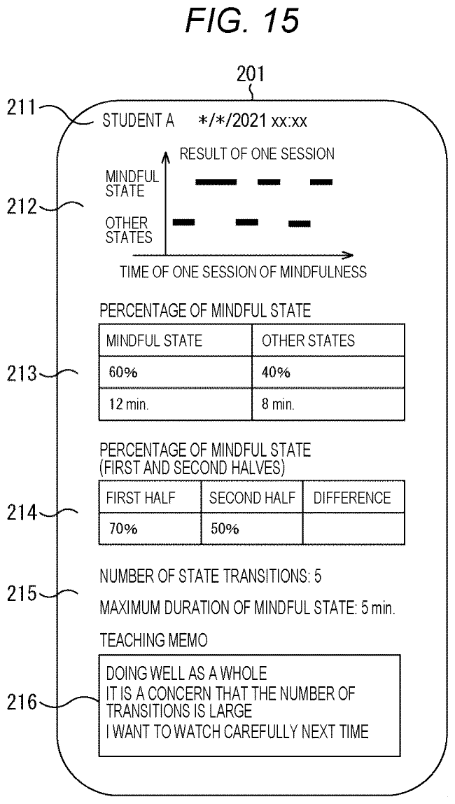

FIG. 14 is a diagram illustrating an example of a screen (referred to as an eighth screen) presented to the student A in a case where there is a teacher, and FIG. 15 is a diagram illustrating an example of a screen (referred to as a ninth screen) presented to the teacher corresponding to the eighth screen.

The eighth screen presented to the student A illustrated in FIG. 14 will be described. Referring to the eighth screen illustrated in FIG. 14, a date display part 191, a thirteenth graph display part 192, a fifth table display part 193, a sixth table display part 194, a third information display part 195, a personal memo input part 196, and a teacher memo display part 197 are provided in this order from the top of the display unit 101.

The eighth screen illustrated in FIG. 14 basically has a configuration similar to that of the first screen illustrated in FIG. 5, and description thereof is omitted as appropriate. Like the date display part 121 and the first graph display part 122 of the first screen (FIG. 5), the date display part 191 and the thirteenth graph display part 192 are areas for displaying a date when learning was performed and a graph indicating a result of the learning at that time.

The fifth table display part 193 and the sixth table display part 194 correspond to the first graph display part 122 and the first table display part 123 (FIG. 5), but are displayed in a minimized state on the screen illustrated in FIG. 14, and tables are not displayed. When expand buttons displayed to the left of text are used, tables similar to those displayed in the first graph display part 122 (FIG. 5) and the first table display part 123 (FIG. 5) are displayed.

As with the first information display part 125 (FIG. 5), the third information display part 195 displays the number of state transitions and the maximum duration of the mindful state.

As with the first memo input part 126 (FIG. 5), the personal memo input part 196 is a field in which the student A takes notes of subjective thoughts, impressions, points to note, and the like.

The teacher memo display part 197 displays memos input by the teacher using the teacher terminal 14. In the example illustrated in FIG. 14, a displayed memo says, “You have gotten better than the previous time. Keep up the good work. It will be even better if you practice to increase your awareness of breathing for the first two minutes”.

The screen to be presented to the student thus includes the personal memo input part 196 to which the student inputs memos and the teacher memo display part 197 in which messages from the teacher are displayed.

### Ninth Screen

The ninth screen presented to the teacher illustrated in FIG. 15 will be described. The ninth screen is a screen displayed on the display unit 201 of the teacher terminal 14.

Referring to the ninth screen illustrated in FIG. 15, a date display part 211, a fourteenth graph display part 212, a seventh table display part 213, an eighth table display part 214, a fourth information display part 215, and a first teacher memo input part 216 are provided in this order from the top of the display unit 201.

The date display part 211 displays a name of the student and a date when the student performed learning.

A graph, tables, and information are displayed in the fourteenth graph display part 212, the seventh table display part 213, the eighth table display part 214, and the fourth information display part 215 as with the thirteenth graph display part 192, the fifth table display part 193, the sixth table display part 194, and the third information display part 195 of the eighth screen for a student (FIG. 14). In the example of the screen illustrated in FIG. 15, the graph and the tables displayed in the fourteenth graph display part 212, the seventh table display part 213, and the eighth table display part 214 are displayed in an expanded state, but may be displayed in a minimized state.

The first teacher memo input part 216 is a field in which the teacher writes, for the student A, memos including subjective evaluations by the teacher such as how much the student A has progressed, what kind of instruction should be given to further progress, points of concern in current learning, and the like. In the example shown in FIG. 15, a written memo says, “Doing well as a whole. It is a concern that the number of transitions is large. I want to watch carefully next time”.

A memo input by the student A may be displayed on the screen for the teacher. In this case, in consideration of privacy, a mechanism may be provided in which, for example, the memo is displayed on the screen for the teacher only in a case where there is an approval of the student A or only memos input by the student A as messages desired to be conveyed to the teacher are displayed.

As described with reference to FIGS. 14 and 15, a screen suitable for a student may be displayed for the student A, and a screen suitable for a teacher may be displayed for the teacher.

### Tenth Screen

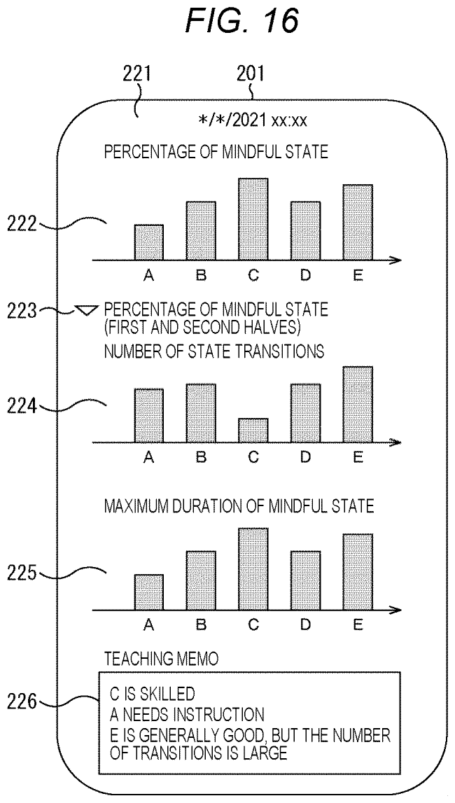

FIG. 16 is a diagram illustrating another example of the screen (referred to as a tenth screen) for a teacher. Examples of screens illustrated in FIG. 16 and subsequent drawings are examples of screens that collectively display information regarding a plurality of students in a case where one teacher is teaching a plurality of students.

In the examples of the screens illustrated in FIG. 16 and the subsequent drawings, A, B, C, D, and E represent the student A, a student B, a student C, a student D, and a student E, respectively, and names, face images, and the like of the students are displayed on an actual screen.

The tenth screen illustrated in FIG. 16 includes, in order from the top, a date display part 221, a fifteenth graph display part 222, a sixteenth graph display part 223, a seventeenth graph display part 224, an eighteenth graph display part 225, and a second teacher memo input part 226.

The date display part 221 displays a date and a time when the plurality of students performed learning and a period of the learning.

The fifteenth graph display part 222 displays a bar graph of percentage of the mindful state of the students A to E. The sixteenth graph display part 223 displays only item names since a graph related to the percentage of the mindful state of the students A to E (first and second halves) is displayed in a minimized state. The graph is displayed by using a mark to the left of text “percentage of mindful state (first and second halves)”.

The seventeenth graph display part 224 displays a bar graph of the number of state transitions of the students A to E. The eighteenth graph display part 225 displays a bar graph of maximum duration of the mindful state of the students A to E.

By referring to these graphs, degrees of progress of the students A to E can be compared and examined. A result of the comparison and examination can be input to the second teacher memo input part 226. In the example illustrated in FIG. 16, a memo input to the second teacher memo input part 226 says, “C is skilled. A needs instruction. E is generally good, but the number of transitions is large”.

### Eleventh Screen

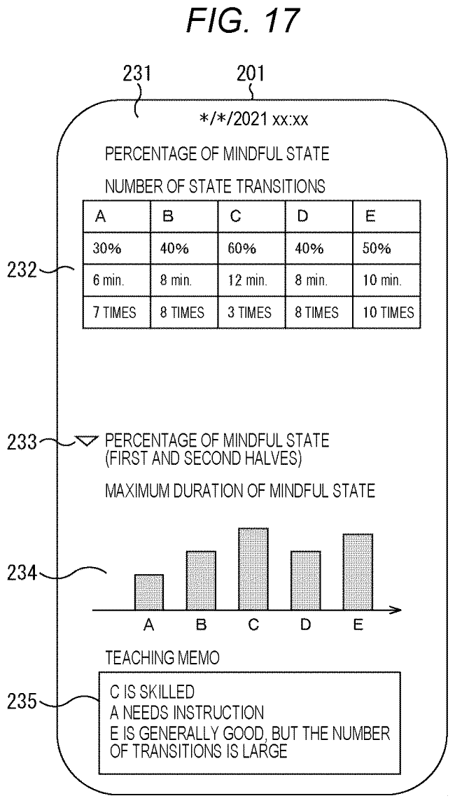

FIG. 17 is a diagram illustrating another example of the screen (referred to as an eleventh screen) for a teacher. The eleventh screen illustrated in FIG. 17 includes a date display part 231, a first table display part 232, a nineteenth graph display part 233, a twentieth graph display part 234, and a third teacher memo input part 235.

The eleventh screen illustrated in FIG. 17 is basically similar to the tenth screen illustrated in FIG. 16, except that the graph related to the percentage of the mindful state displayed in the fifteenth graph display part 222 and the graph related to the number of transitions displayed on the seventeenth graph display part 224 are collectively displayed as a table in the first table display part 232.

The first table display part 232 displays a table in which the percentage of the mindful state, a total time of the mindful state, and the number of state transitions of each of the students A to E are associated with one another.

Results of learning by a plurality of students may thus be displayed in a table. The teacher may be able to select whether to display a table, a graph, or both a table and a graph.

### Twelfth Screen

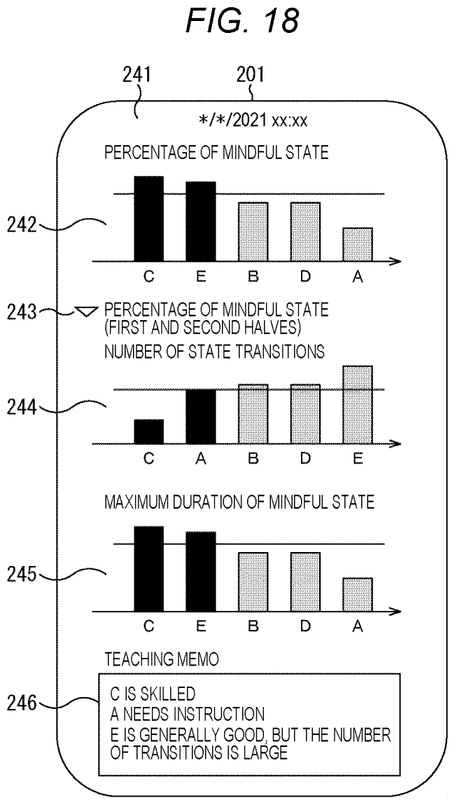

FIG. 18 is a diagram illustrating another example of the screen (referred to as a twelfth screen) for a teacher. The twelfth screen illustrated in FIG. 18 includes a date display part 241, a twenty-first graph display part 242, a twenty-second graph display part 243, a twenty-third graph display part 244, a twenty-fourth graph display part 245, and a fourth teacher memo input part 246.

The twelfth screen illustrated in FIG. 18 has a configuration similar to that of the tenth screen illustrated in FIG. 16, but is different in that the displayed graphs are arranged in order of the degree of progress.

The twenty-first graph display part 242 displays a bar graph of the percentage of the mindful state of the students A to E. The bar graph is arranged in order of the degree of progress. In the example illustrated in FIG. 18, the student C, the student E, the student B, the student D, and the student A are arranged in this order.

In the twenty-first graph display part 242, a standard line indicating a standard for the degree of progress is displayed, and it can be recognized at a glance that students whose bars extend beyond the standard line have reached a predetermined degree of progress. The standard for the degree of progress may be the percentage of the mindful state of 50% or more, and the standard line is displayed at a position corresponding to 50%.

Although 50% is described as an example of the standard here, the standard is not limited to 50%, and another standard may be provided, instead. Furthermore, for example, a mechanism in which the teacher can set any standard may be provided.

In the example illustrated in FIG. 18, it can be seen that the students C and E have reached the standard degree of progress. Bars of students who have reached the standard degree of progress may be displayed differently from bars of students who have not reached the standard degree of progress, that is, for example, may be displayed in a different color.

The twenty-second graph display part 243 displays only item names since a graph related to the percentage of the mindful state of the students A to E (first and second halves) is displayed in a minimized state. The graph is displayed by using a mark to the left of text “percentage of mindful state (first and second halves)”.

The twenty-third graph display part 244 displays a bar graph of the number of state transitions of the students A to E. This bar graph is also arranged in order of the degree of progress, and in the example illustrated in FIG. 18, the student C, the student A, the student B, the student D, and the student E are arranged in this order.

In the twenty-third graph display part 244, a standard line indicating a standard for the degree of progress is displayed, and it can be recognized at a glance that students whose bars are below the standard line have reached a predetermined degree of progress. The standard for the degree of progress may be the number of state transitions of six or less, and the standard line is displayed at a position corresponding to six times. In the example illustrated in FIG. 18, it can be seen that the number of state transitions of the student C and the student A is less than the standard and the student C and the student A have reached the standard degree of progress.

Although six times is described as an example of the standard here, the standard is not limited to six times, and another standard may be provided, instead. Furthermore, for example, a mechanism in which the teacher can set any standard may be provided.

The twenty-fourth graph display part 245 displays a bar graph of the maximum duration of the mindful state of the students A to E. This bar graph is also arranged in order of the degree of progress, and in the example illustrated in FIG. 18, the student C, the student E, the student B, the student D, and the student A are arranged in this order.

In the twenty-fourth graph display part 245, a standard line indicating a standard for the degree of progress is displayed, and it can be recognized at a glance that students whose bars extend beyond the standard line have reached a predetermined degree of progress. The standard for the degree of progress may be the maximum duration of the mindful state of 5 minutes or more, and the standard line is displayed at a position corresponding to 5 minutes. In the example illustrated in FIG. 18, it can be seen that duration of the mindful state of the student C and the student E is long and the student C and the student E have reached the standard degree of progress.

Although 5 minutes is described as an example of the standard here, the standard is not limited to 5 minutes, and another standard may be provided, instead. Furthermore, for example, a mechanism in which the teacher can set any standard may be provided.

By displaying students side by side in order of the degree of progress for each parameter like this, it is possible to perform comparison and examination separately for persons with high skill and persons with low skill. A result of the comparison and examination can be input to the fourth teacher memo input part 246.

### Thirteenth Screen

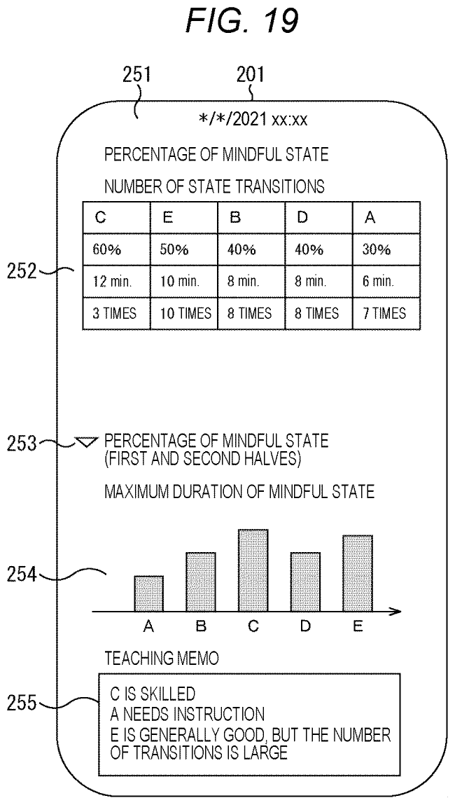

FIG. 19 is a diagram illustrating another example of the screen (referred to as a thirteenth screen) for a teacher. The thirteenth screen illustrated in FIG. 19 includes a date display part 251, a second table display part 252, a twenty-fifth graph display part 253, a twenty-sixth graph display part 254, and a fifth teacher memo input part 255.

The thirteenth screen illustrated in FIG. 19 has a configuration similar to that of the eleventh screen illustrated in FIG. 17, but is different in that the displayed table arranged in order of the degree of progress.

The second table display part 252 displays a table in which the percentage of the mindful state, the total time of the mindful state, and the number of state transitions of the students A to E are associated with one another and in which values are arranged in order of the degree of progress of the students. In the example illustrated in FIG. 19, the students A to E are arranged in descending order of the percentage of the mindful state, but the students A to E may be arranged according to the total time of the mindful state or the number of state transitions.

In the example illustrated in FIG. 19, since the degree of progress, that is, the percentage of the mindful state in this case, is higher in order of the student C, the student E, the student B, the student D, and the student A, the parameters are displayed in this order in association with one another.

Although an example in which the graph related to the maximum duration of the mindful state displayed in the twenty-sixth graph display part 254 is not displayed in order of the degree of progress has been described in the example illustrated in FIG. 19, the graph may be displayed in order of the degree of progress as in the graph displayed in the twenty-fourth graph display part 245 illustrated in FIG. 18, instead. Alternatively, a table may be displayed instead of a graph.

### Fourteenth Screen

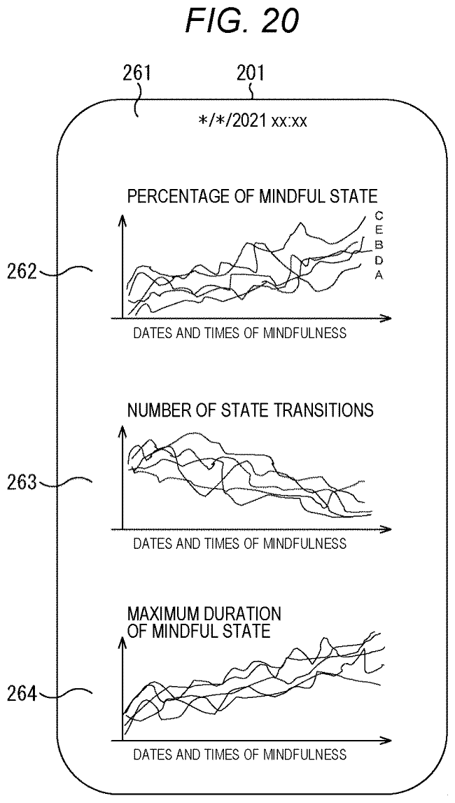

FIG. 20 is a diagram illustrating another example of the screen (referred to as a fourteenth screen) for a teacher. The fourteenth screen illustrated in FIG. 20 includes a date display part 261, a twenty-seventh graph display part 262, a twenty-eighth graph display part 263, and a twenty-ninth graph display part 264.

In graphs displayed on the fourteenth screen illustrated in FIG. 20, line graphs indicating degrees of progress of a plurality of students, that is, the students A to E in this case, are superimposed upon one another.

The twenty-seventh graph display part 262 displays a graph in which line graphs of the percentage of the mindful state of the students A to E are superimposed upon one another. Since a graph of the percentage of the mindful state is better as a value increases, a graph in which a value increases over time indicates that a student has been making progress.

The twenty-eighth graph display part 263 displays a graph in which line graphs of the number of state transitions of the students A to E are superimposed are superimposed upon one another. Since a graph of the number of state transitions is better as a value decreases, a graph in which a value decreases over time indicates that a student has been making progress.

The twenty-ninth graph display part 264 displays a graph in which line graphs of the maximum duration of the mindful state of the students A to E are superimposed upon one another. Since a graph of the maximum duration of the mindful state is better as a value increases, a graph in which a value increases over time indicates that a student has been making progress.

By viewing these graphs, the teacher can examine the degrees of progress of the students while comparing the students. For example, it can be seen that a student whose graph includes a steeper slope has progressed (become proficient) faster. On the basis of obtained information, the teacher can also input memos. Although not illustrated in FIG. 20, a teacher memo input part may be provided, and a memo can be input to the input part. In screens described below, too, a teacher memo input part is provided so that the teacher can leave memos, even if the teacher memo input part is not illustrated.

### Fifteenth Screen

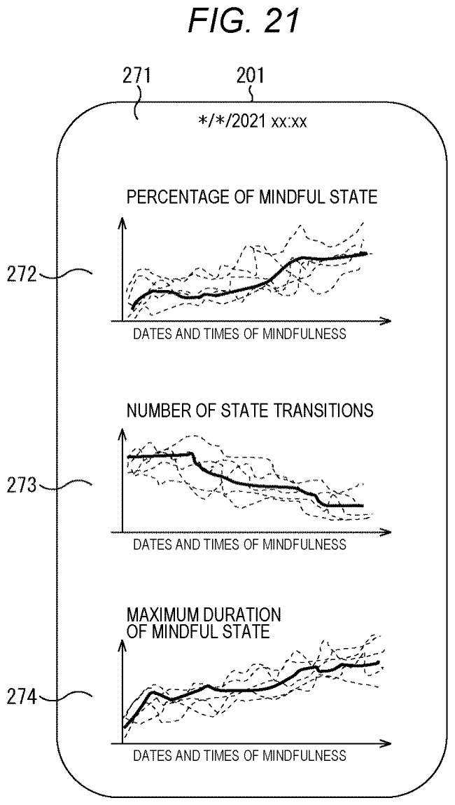

FIG. 21 is a diagram illustrating another example of the screen (referred to as a fifteenth screen) for a teacher. The fifteenth screen illustrated in FIG. 21 includes a date display part 271, a thirtieth graph display part 272, a thirty-first graph display part 273, and a thirty-second graph display part 274.

Graphs displayed on the fifteenth screen illustrated in FIG. 21 are similar to the graphs displayed on the fourteenth screen illustrated in FIG. 20 in that line graphs indicating the degrees of progress of a plurality of students, that is, the students A to E in this case, are superimposed upon one another, and is different in that a reference graph such as a graph of average values or a graph of median values is additionally displayed on the graphs.

The thirtieth graph display part 272 displays a graph in which line graphs of the percentage of the mindful state of the students A to E are superimposed upon one another, and a graph representing average values, for example, is also superimposed.

The thirty-first graph display part 273 displays a graph in which line graphs of the number of state transitions of the students A to E are superimposed upon one another, and a graph representing average values, for example, is also superimposed.

The thirty-second graph display part 274 displays a graph in which line graphs of the maximum duration of the mindful state of the students A to E are superimposed upon one another, and a graph representing average values, for example, is also superimposed.

Graphs of students thus need not be simply displayed, and a reference graph may also be superimposed upon the graphs of the students. By superimposing such a reference graph, degrees of progress of students can be more easily compared and examined.

### Sixteenth Screen

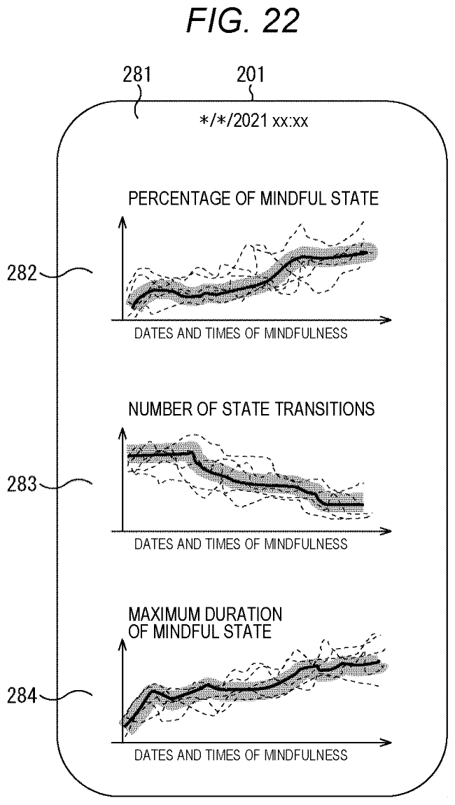

FIG. 22 is a diagram illustrating another example of the screen (referred to as a sixteenth screen) for a teacher. The sixteenth screen illustrated in FIG. 22 includes a date display part 281, a thirty-third graph display part 282, a thirty-fourth graph display part 283, and a thirty-fifth graph display part 284.

Graphs displayed on the sixteenth screen illustrated in FIG. 22 are similar to the graphs displayed on the fifteenth screen illustrated in FIG. 21 in that line graphs indicating the degrees of progress of a plurality of students, that is, the students A to E in this case, and a reference graph such as a graph of average values or a graph of median values are superimposed upon one another, and is different in that a graph in which a range of variation such as a quartile range or a standard deviation is further added is displayed on the graphs.

The thirty-third graph display part 282 displays a graph in which line graphs of the percentage of the mindful state of the students A to E are superimposed upon one another. For example, a graph representing average values is superimposed, and a range of variation such as a standard deviation of the average values is superimposed upon the graph representing the average values.

The thirty-fourth graph display part 283 displays a graph in which line graphs of the number of state transitions of the students A to E are superimposed upon one another. For example, a graph representing average values is superimposed, and a range of variation such as a standard deviation of the average values is superimposed upon the graph representing the average values.

The thirty-fifth graph display part 284 displays a graph in which line graphs of the maximum duration of the mindful state of the students A to E are superimposed upon one another. For example, a graph representing average values is superimposed, and a range of variation such as a standard deviation of the average values is superimposed upon the graph representing the average values.

Graphs of students thus need not be simply displayed, and a reference graph and an indicator of a degree of variation of the reference graph may also be superimposed upon the graphs of the students. By superimposing such a reference graph, degrees of progress of students can be more easily compared and examined.

### Seventeenth Screen

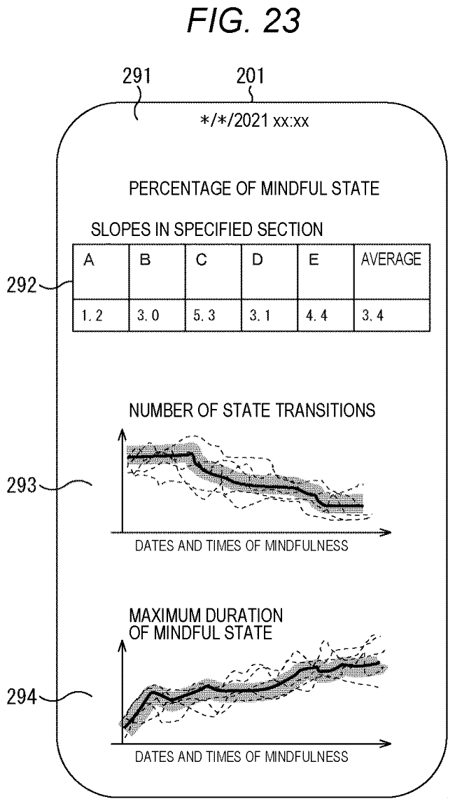

FIG. 23 is a diagram illustrating another example of the screen (referred to as a seventeenth screen) for a teacher. The seventeenth screen illustrated in FIG. 23 includes a date display part 291, a first slope table display part 292, a thirty-sixth graph display part 293, and a thirty-seventh graph display part 294.

The seventeenth screen illustrated in FIG. 23 includes the first slope table display part 292 and displays a table regarding slopes. In the example illustrated in FIG. 23, for example, a slope of the graph of the percentage of the mindful state of each student displayed in the thirty-third graph display part 282 in FIG. 22 is displayed as the table in the first slope table display part 292. This slope is a slope of the graph in a specified section (period). The section may be specified by the teacher or may be set in advance. Alternatively, for example, a table in which the steepest slopes of graphs of certain students is described may be displayed instead of slopes in a predetermined section.

In the example of the first slope table display part 292 illustrated in FIG. 23, a table describing that the slope of the student A is 1.2, the slope of the student B is 3.0, the slope of the student C is 5.3, the slope of the student D is 3.1, and the slope of the student E is 4.4, and an average value of these is 3.4 is displayed. The steeper the slope, the higher the degree of progress.

Although an example in which the same graph as that displayed in the thirty-fourth graph display part 293 illustrated in FIG. 22 is displayed in the thirty-sixth graph display part 293 illustrated in FIG. 23 has been described, a table in which a slope of the graph of the number of state transitions is described for each student may be displayed as in the first slope table display part 292, instead.

Although an example in which the same graph as that displayed in the thirty-fifth graph display part 294 illustrated in FIG. 22 is displayed in the thirty-seventh graph display part 294 illustrated in FIG. 23 has been described, a table in which a slope of the graph of the maximum duration of the mindful state is described for each student may be displayed as in the first slope table display part 292, instead.

Not only graphs but also information with which students can be compared using a table may thus be presented to the teacher. A mechanism for enabling the teacher to select whether to display a graph or a table may be provided.

### Eighteenth Screen

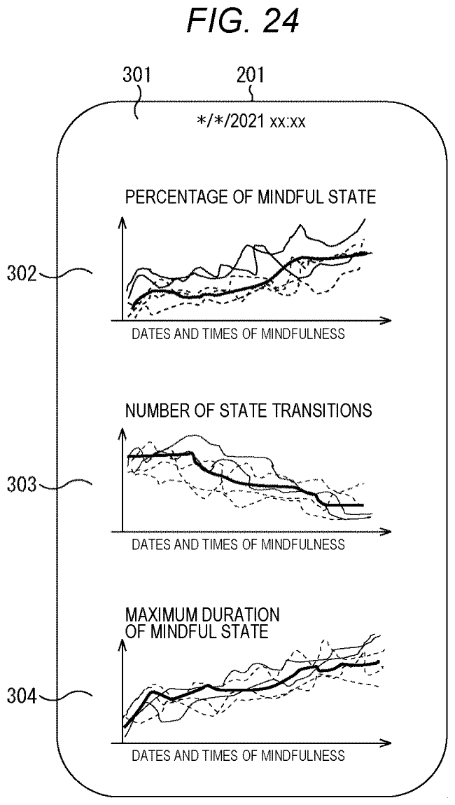

FIG. 24 is a diagram illustrating another example of the screen (referred to as an eighteenth screen) for a teacher. The eighteenth screen illustrated in FIG. 24 includes a date display part 301, a thirty-eighth graph display part 302, a thirty-ninth graph display part 303, and a fortieth graph display part 304.

The eighteenth screen illustrated in FIG. 24 is the same as the fifteenth screen illustrated in FIG. 21, but is different from the fourteenth screen in that graphs exceeding a standard among displayed graphs are displayed differently from other graphs not exceeding the standard. The graphs exceeding the standard can be said to be graphs in which the degree of progress is higher than or equal to a certain level.

The thirty-eighth graph display part 302 displays a graph in which line graphs of the percentage of the mindful state of the students A to E are superimposed upon one another, and displays two of the graphs (graphs indicated by solid lines in the figure) differently from the other three graphs (graphs indicated by dotted lines in the figure) because the two graphs exceed the standard.

Since a graph of the percentage of the mindful state is better as a value increases, graphs larger than average values or median values are displayed in color, for example, as graphs of students with high degrees of progress. In the example illustrated in FIG. 24, the two graphs exceeding the standard are indicated by the solid lines, and the other three graphs are indicated by the dotted lines. A thick solid line is a graph representing average values or median values.

The thirty-ninth graph display part 303 displays a graph in which line graphs of the number of state transitions of the students A to E are superimposed upon one another, and displays two of the graphs differently from the other three graphs because the two graphs exceed the standard. Since a graph of the number of state transitions is better as a value decreases, a graph smaller than average values or median values are displayed in color, for example, as graphs of students with high degrees of progress.

The fortieth graph display part 304 displays a graph in which line graphs of the maximum duration of the students A to E are superimposed upon one another, and displays two of the graphs differently from the other three graphs because the two graphs exceed the standard. Since a graph of the maximum duration of the mindful state is better as a value increases, graphs larger than average values or median values are displayed in color, for example, as graphs of students with high degrees of progress.

Graph of students who are rapidly making progress and graphs of students who are not making progress rapidly may thus be displayed in such a way as to be recognized at a glance. With such display, it is possible to separately compare and examine students who are rapidly making progress and students who are not making progress rapidly.

Note that, in the eighteenth screen illustrated in FIG. 24, as in the sixteenth screen illustrated in FIG. 22, a graph in which an area representing variation is superimposed upon a graph of average values or median values may be displayed.

### Nineteenth Screen

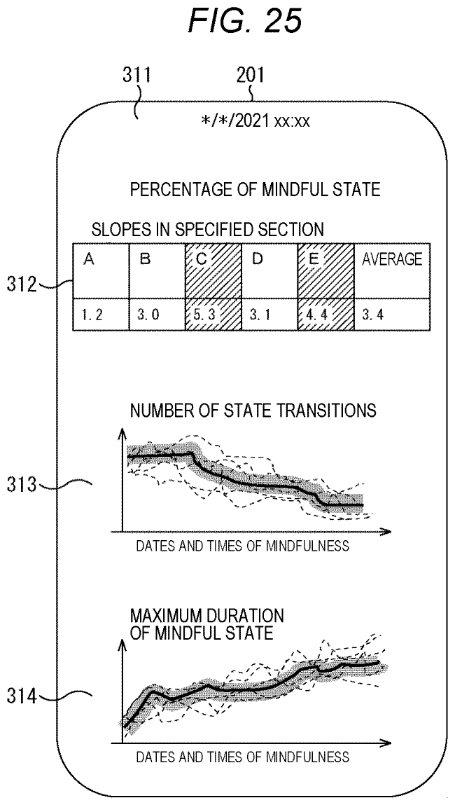

FIG. 25 is a diagram illustrating another example of the screen (referred to as a nineteenth screen) for a teacher. The nineteenth screen illustrated in FIG. 25 includes a date display part 311, a second slope table display part 312, a forty-first graph display part 313, and a forty-second graph display part 314.

The nineteenth screen illustrated in FIG. 25 is the same as the seventeenth screen illustrated in FIG. 23, but is different in that slopes exceeding a standard value and corresponding student names are displayed differently from slopes not exceeding the standard value and corresponding student names in the table regarding slopes displayed in the second slope table display part 312.

In the second slope table display part 312 illustrated in FIG. 25, since a slope of 5.3 of the student C and a slope of 4.4 of the student E exceed the standard value of 3.5, for example, fields corresponding to the students C and E are displayed, for example, in color.

Although an example in which graphs displayed in the forty-first graph display part 313 and the forty-second graph display part 314 are the same as those displayed in the thirty-sixth graph display part 293 and the thirty-seventh graph display part 294 in FIG. 23 has been described in the example illustrated in FIG. 25, graphs exceeding the standard value may be displayed differently from graphs not exceeding the standard value as with the graphs displayed in the thirty-ninth graph display part 303 and the fortieth graph display part 304 in FIG. 24.

Although an example in which a graph is displayed in each of the forty-first graph display part 313 and the forty-second graph display part 314 has been described in the example illustrated in FIG. 25, a table regarding slopes may be displayed like the table displayed in the second slope table display part 312, instead. A table regarding slopes may be displayed, and slopes exceeding the standard value and student names corresponding to the slopes may be displayed differently from other slopes and corresponding student names.

According to the present technology, it is possible to provide information for a student and/or a teacher to objectively evaluate a state during mindfulness. For example, visualized information regarding a mindful state can be provided, and a state during mindfulness can be objectively evaluated by viewing the provided visualized information. By viewing the visualized information regarding the mindful state, the student can quantitatively look back and use the information for progress over time.

For example, the student can set an achievement value within a predetermined period, and determine whether or not the set achievement value has been achieved by viewing the visualized information. Furthermore, it is easy for the teacher to perform processing, such as certifying a student whose degree of achievement has reached a predetermined value as a teacher, by viewing the visualized information.

As the visualized information regarding the mindful state, for example, information can be provided with which it is possible to quantitatively know length of the states during mindfulness (the mindful state focused on breathing and the other states), length of time for which mindfulness is performed, the number of state transitions, and the like. A result of an estimated state can be visualized, temporal states can be visualized, and a proficiency level can be defined and visualized.

It is possible to estimate the mindful state from brain waves, respiration, and the like, which are easily measured from the outside, and simply evaluate the mindful state.

Real-time feedback can be performed during learning of mindfulness, and a student can be urged to return to the mindful state to maintain the mindful state. For example, in a case where a student gets out of a state of being focused on breathing (or in a case where a student is about to get out of the state of being focused on breathing), feedback can be performed in real-time using voice, vibration, or the like, and the student can be urged to return to the state of being focused on breathing.

Note that the program executed by the computer may be a program in which processing is performed in time series in the order described in the present specification or may be a program in which processing is performed in parallel or at necessary timing such as when a call is made.

In the present specification, the system represents the entire device including a plurality of devices.

Note that the effects described in the present description are merely examples and are not limited, and other effects may be provided.

Note that the embodiments of the present technology are not limited to the above-described embodiments, and various changes can be made without departing from the gist of the present technology.

Note that the present technology can also have the following configurations.
(1)

An information processing apparatus including:
an obtaining unit that obtains biological information regarding a user;
a detection unit that analyzes the biological information obtained by the obtaining unit and that detects a mindful state of the user; and
a generation unit that generates image data regarding a screen for presenting information regarding the mindful state detected by the detection unit to the user.
(2)

The information processing apparatus according to (1), in which
the screen includes a graph indicating time for which the mindful state remained established and time for which the mindful state was not established.
(3)

The information processing apparatus according to (1) or (2), in which
the screen includes a screen that presents percentage of time for which the mindful state remained established in a predetermined period.
(4)

The information processing apparatus according to any one of (1) to (3), in which
the screen includes a screen that presents percentage of time for which the mindful state remained established in a first half and a second half of a predetermined period.
(5)

The information processing apparatus according to any one of (1) to (4), in which
the screen includes a screen that presents a number of transitions between the mindful state and a state other than the mindful state.
(6)

The information processing apparatus according to any one of (1) to (5), in which
the screen includes a screen that presents maximum duration of the mindful state.
(7)

The information processing apparatus according to any one of (1) to (6), in which
the screen includes a screen that displays a moving image obtained by shooting the user within measurement time in which the biological information was measured.
(8)

The information processing apparatus according to (7), in which
the moving image is a moving image obtained by shooting one of the mindful state, the state other than the mindful state, and a state of transition between the mindful state and the mindful state.
(9)

The information processing apparatus according to any one of (1) to (8), in which
the screen includes a field in which the user writes a memo regarding learning of mindfulness.
(10)

The information processing apparatus according to any one of (1) to (9), in which
the screen includes a screen that presents a level of the user in learning of mindfulness.
(11)

The information processing apparatus according to any one of (1) to (10), in which
the screen includes a graph in which percentage of time for which the mindful state remained established in a plurality of periods is arranged in time series.
(12)

The information processing apparatus according to any one of (1) to (11), in which
the screen includes a screen for a teacher who teaches mindfulness to the user, who is a student, the screen for the teacher presenting percentage of time of a plurality of students for which the mindful state remained established in learning time.
(13)

The information processing apparatus according to (1), in which
the screen includes a screen for a teacher who teaches mindfulness to the user, who is a student, the screen for the teacher presenting a number of transitions of a plurality of students between the mindful state and a state other than the mindful state.
(14)

The information processing apparatus according to (1) or (13), in which
the screen includes a screen for a teacher who teaches mindfulness to the user, who is a student, the screen for the teacher presenting maximum duration of the mindful state of a plurality of students.
(15)

The information processing apparatus according to (1), (13), or (14), in which
the screen includes a screen for a teacher who teaches mindfulness to the user, who is a student, and a graph of percentage of time of the mindful state of a plurality of students for which the mindful state remained established in learning time is superimposed upon the screen for the teacher.
(16)

The information processing apparatus according to any one of (1) and (13) to (15), in which
the screen includes a screen for a teacher who teaches mindfulness to the user, who is a student, and a graph of a number of transitions of a plurality of students between the mindful state and a state other than the mindful state is superimposed upon the screen for the teacher.
(17)

The information processing apparatus according to any one of (1) and (13) to (16), in which
the screen includes a screen for a teacher who teaches mindfulness to the user, who is a student, and a graph of maximum duration of the mindful state of a plurality of students is superimposed upon the screen for the teacher.
(18)

The information processing apparatus according to any one of (1) and (13) to (17), in which
the screen includes a screen for a teacher who teaches mindfulness to the user, who is a student, the screen for the teacher including a field in which the teacher writes a memo regarding the teaching for the learning.
(19)

An information processing method used by an information processing apparatus, the information processing method including:
obtaining biological information regarding a user;
analyzing the obtained biological information and detecting a mindful state of the user; and
generating image data regarding a screen for presenting information regarding the detected mindful state to the user.
(20)

A program for causing a computer to execute a process including the steps of:
obtaining biological information regarding a user;
analyzing the obtained biological information and detecting a mindful state of the user; and
generating image data regarding a screen for presenting information regarding the detected mindful state to the user.

### REFERENCE SIGNS LIST

1 Information processing system
11 Student terminal
12 Measurement apparatus
13 Information processing apparatus
14 Teacher terminal
15 Network
61 Communication control unit
62 Measurement information obtaining unit
63 Analysis unit
64 Image data generation unit
101 Display unit
122 First graph display part
123 First table display part
124 Second table display part
125 First information display part
126 First memo input part
134 First moving image display part
152 Level display part
196 Personal memo input part
197 Teacher memo display part
201 Display unit
212 Fourteenth graph display part
213 Seventh table display part
214 Eighth table display part
215 Fourth information display part
216 First teacher memo input part
232 First table display part
292 First slope table display part

1. An information processing apparatus comprising:
an obtaining unit that obtains biological information regarding a user;
a detection unit that analyzes the biological information obtained by the obtaining unit and that detects a mindful state of the user; and
a generation unit that generates image data regarding a screen for presenting information regarding the mindful state detected by the detection unit to the user.

2. The information processing apparatus according to claim 1, wherein
the screen includes a graph indicating time for which the mindful state remained established and time for which the mindful state was not established.

3. The information processing apparatus according to claim 1, wherein
the screen includes a screen that presents percentage of time for which the mindful state remained established in a predetermined period.

4. The information processing apparatus according to claim 1, wherein
the screen includes a screen that presents percentage of time for which the mindful state remained established in a first half and a second half of a predetermined period.

5. The information processing apparatus according to claim 1, wherein
the screen includes a screen that presents a number of transitions between the mindful state and a state other than the mindful state.

6. The information processing apparatus according to claim 1, wherein
the screen includes a screen that presents maximum duration of the mindful state.

7. The information processing apparatus according to claim 1, wherein
the screen includes a screen that displays a moving image obtained by shooting the user within measurement time in which the biological information was measured.

8. The information processing apparatus according to claim 7, wherein
the moving image is a moving image obtained by shooting one of the mindful state, the state other than the mindful state, and a state of transition between the mindful state and the mindful state.

9. The information processing apparatus according to claim 1, wherein
the screen includes a field in which the user writes a memo regarding learning of mindfulness.

10. The information processing apparatus according to claim 1, wherein
the screen includes a screen that presents a level of the user in learning of mindfulness.

11. The information processing apparatus according to claim 1, wherein
the screen includes a graph in which percentage of time for which the mindful state remained established in a plurality of periods is arranged in time series.

12. The information processing apparatus according to claim 1, wherein
the screen includes a screen for a teacher who teaches mindfulness to the user, who is a student, the screen for the teacher presenting percentage of time of a plurality of students for which the mindful state remained established in learning time.

13. The information processing apparatus according to claim 1, wherein
the screen includes a screen for a teacher who teaches mindfulness to the user, who is a student, the screen for the teacher presenting a number of transitions of a plurality of students between the mindful state and a state other than the mindful state.

14. The information processing apparatus according to claim 1, wherein
the screen includes a screen for a teacher who teaches mindfulness to the user, who is a student, the screen for the teacher presenting maximum duration of the mindful state of a plurality of students.

15. The information processing apparatus according to claim 1, wherein
the screen includes a screen for a teacher who teaches mindfulness to the user, who is a student, and a graph of percentage of time of the mindful state of a plurality of students for which the mindful state remained established in learning time is superimposed upon the screen for the teacher.

16. The information processing apparatus according to claim 1, wherein
the screen includes a screen for a teacher who teaches mindfulness to the user, who is a student, and a graph of a number of transitions of a plurality of students between the mindful state and a state other than the mindful state is superimposed upon the screen for the teacher.

17. The information processing apparatus according to claim 1, wherein
the screen includes a screen for a teacher who teaches mindfulness to the user, who is a student, and a graph of maximum duration of the mindful state of a plurality of students is superimposed upon the screen for the teacher.

18. The information processing apparatus according to claim 1, wherein
the screen includes a screen for a teacher who teaches mindfulness to the user, who is a student, the screen for the teacher including a field in which the teacher writes a memo regarding the teaching for the learning.

19. An information processing method used by an information processing apparatus, the information processing method comprising:
obtaining biological information regarding a user;
analyzing the obtained biological information and detecting a mindful state of the user; and
generating image data regarding a screen for presenting information regarding the detected mindful state to the user.

20. A program for causing a computer to execute a process comprising the steps of:
obtaining biological information regarding a user;
analyzing the obtained biological information and detecting a mindful state of the user; and
generating image data regarding a screen for presenting information regarding the detected mindful state to the user.
